(19) United States
(12) Patent Application Publication
(10) Pub. No.: US 2008/0097314 A1
(43) Pub. Date: Apr. 24, 2008

(54) Safety syringe

(76) Inventors: Jih-Hsiung Yang, Wufeng Hsiang (TW); Hsiu-Chih Lin, Wufeng Hsiang (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 11/500,384

(22) Filed: Aug. 8, 2006

(51) Int. Cl. A61M 1/00; A61M 25/00

(52) U.S. Cl. 604/122; 604/264

(57) ABSTRACT

A safety syringe includes a barrel, a needle assembly and a plunger. The barrel has at least one protrusion formed on one end of the barrel. The needle assembly includes a hub and a needle. The hub has at least one L-shaped groove respectively corresponding to and engaging with the at least one protrusion of the barrel. The needle is attached on the hub. The plunger is mounted slidably in the barrel. Accordingly, the needle assembly can be retracted into the barrel to keep any person from being injured by a used needle and is easily manufactured.

### BACKGROUND OF THE INVENTION

1. Field of Invention

The present invention relates to a syringe, and more particularly to a safety syringe that is easy to be manufactured.

2. Description of the Related Art

Injections of drugs and blood with a traditional syringe are common health care procedure. But the traditional syringe can not retract its needle and may hurt persons easily. The used syringe may transmit hepatitis B virus (HBV), hepatitis C virus (HCV) and human immunodeficiency virus (HIV). To avoid hurting persons, a conventional safe syringe with a retractable needle is provided.

However, the conventional safety syringe has a complicated structure and is hard to be manufactured. So the conventional safety syringe is expensive and is not extensively used. In addition, an inner thread is always necessary for a conventional safety syringe, but to form an inner thread in a conventional safety syringe is trouble.

To overcome the shortcomings, the present invention provides a safety syringe to mitigate or obviate the aforementioned problems.

### SUMMARY OF THE INVENTION

The primary objective of the present invention is to provide a safety syringe that is safe to use and easily manufactured.

A safety syringe in accordance with the present invention comprises a barrel, a needle assembly and a plunger.

The barrel has a proximal open end, a distal open end, an inner wall, an inner diameter; at least one protrusion and a flange. The protrusion is formed on the inner wall near the proximal open end of the barrel. The flange is formed on the distal open end of the barrel.

The needle assembly is mounted on the proximal open end of the barrel and comprises a hub and a needle. The hub have a proximal end, a distal end, an outer surface, at least one L-shaped groove, a connecting stub, a recess and a passage. The at least one L-shaped groove is defined in the outer surface of the hub and respectively corresponds to and engages with the protrusion of the barrel. The connecting stub is formed on the surface of the proximal end of the hub and protruded out of the proximal open end of the barrel. The recess is defined in the distal end of the hub. The passage is defined longitudinally through the hub. The needle is attached on the connecting stub and communicates with the passage.

The plunger is mounted slidably in the barrel and comprises a push rod and a piston. The push rod has a proximal end and a distal end. The piston is mounted on the proximal end of the push rod and has a diameter, an outer surface, an end facing the hub and a protrusion. The diameter is slightly larger than the inner diameter of the barrel. The protrusion is formed on the end facing the hub and corresponds to the recess of the hub.

Other objectives, advantages and novel features of the invention will become more apparent from the following detailed description when taken in conjunction with the accompanying drawings.

### BRIEF DESCRIPTION OF THE DRAWINGS

FIG. 1 is an exploded perspective view of a safety syringe in accordance with the present invention;

FIG. 2 is an operational cross sectional side view of the safety syringe in FIG. 1 with the plunger being pulled back;

FIG. 3 is an operational cross sectional side view of the safety syringe in FIG. 1 with the plunger being pushed;

FIG. 4 is an operational cross sectional side view of the safety syringe in FIG. 1 with the piston abutting against the hub;

FIG. 5 is an operational cross sectional side view of the safety syringe in FIG. 1 with the hub being loosened;

FIG. 6 is an operational cross sectional side view of the safety syringe in FIG. 1 with the plunger being broken;

FIG. 7 is a perspective view of an alternative embodiment of an integral needle assembly in accordance with the present invention; and

FIG. 8 is a perspective view of an alternative embodiment of an integral plunger in accordance with the present invention.

### DETAILED DESCRIPTION OF THE INVENTION

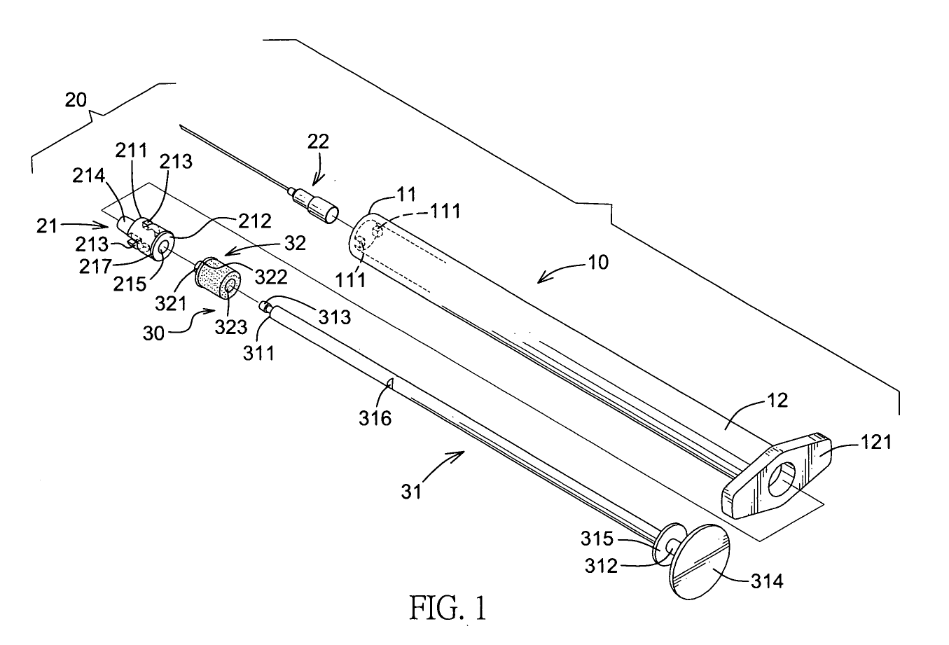

With reference to FIGS. 1, 2 and 3, a safety syringe in accordance with the present invention comprises a barrel (10), a needle assembly (20) and a plunger (30).

The barrel (10) has a proximal open end (11), a distal open end (12), an inner wall, an inner diameter, two protrusions (111) and a flange (121). The protrusions (111) are formed on the inner wall near the proximal open end (11) of the barrel (10) and opposite to each other. The flange (121) is formed on the distal open end (12) of the barrel (10).

The needle assembly (20) is mounted on the proximal open end (11) of the barrel (10) and comprises a hub (21) and a needle (22). The hub (21) is cylindrical and connected to the proximal open end (11) of the barrel (10). The hub (21) has a proximal end (211), a distal end (212), an outer surface, two L-shaped grooves (213), a connecting stub (214), a recess (215), an optional annular lip (217) and a passage (218). The L-shaped grooves (213) are defined in the outer surface of the hub (21), correspond to the protrusions (111) of the barrel (10) and each has a longitudinal part. The longitudinal part forms an opening on the proximal end of the hub (21). The hub (21) is inserted into the barrel (10) from the distal open end (12) till the protrusions (111) of the barrel (10) are placed into the L-shaped grooves (111) from the opening on the proximal end (211) of the hub (21). Then the hub (21) is rotated and locked by the protrusions (111) of the barrel (10), which prevents the hub (21) from moving longitudinally.

The connecting stub (214) is conical and hollow, is formed on the proximal end (211) and protrudes out of the proximal open end (11) of the barrel (10). The recess (215) is conical, is defined in the distal end (212) and has an inner wall. The annular lip (217) is formed on the outer surface of the hub (21) to press against the inner wall of the barrel (10) tightly. The passage (218) is defined longitudinally through the hub (21).

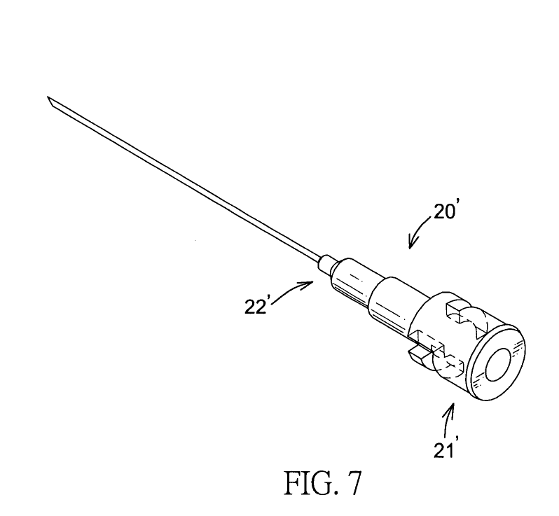

The needle (22) is attached on the connecting stub (214) of the hub (21) and communicates with the passage (218). With further reference to FIG. 7, the needle (22′) can also be integrally formed on the hub (21′) to form an integral needle assembly.

The plunger (30) is mounted slidably in the barrel (10) and comprises a push rod (31) and a piston (32). The push rod (31) has a proximal end (311), a distal end (312), a blocking protrusion (313), a stop (314), a finger tab (315) and an optional notch (316). The blocking protrusion (313) is cylindrical, is formed on the proximal end (311) of the push rod (31) and has an enlarged end. The stop (314) and the finger tab (315) are formed separately on the distal end (312) of the push rod (31) for facilely positioning and pushing the plunger (30). The notch (316) is defined in the push rod (31) to allow the push rod (31) to be easily broken.

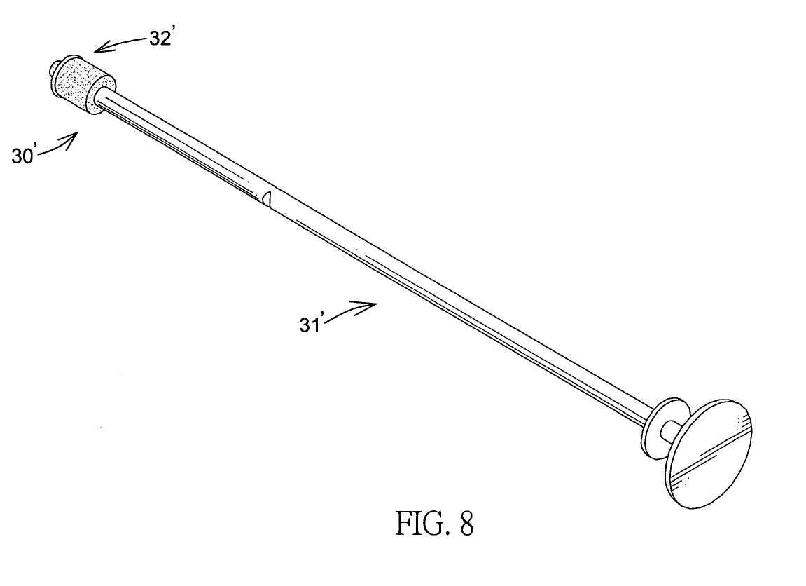

The piston (32) is cylindrical and hollow, is mounted on the proximal end (311) of the push rod (31) and has a diameter, an outer surface, an end facing the hub (21), a protrusion (321) having an outer surface, an optional annular shoulder (322) and a through hole (323). The diameter of the piston (32) is larger slightly than the inner diameter of the barrel (10), so the outer surface of the piston (32) can abut against the inner wall of the barrel (10). The protrusion (321) is formed on the end facing the hub (21) and corresponds to and engages with the recess (215) of the hub (21). The annular shoulder (322) is formed on the outer surface of the piston (32) and presses against the inner wall of the barrel (10) tightly. The through hole (323) is defined through the piston (32) and corresponds to and is mounted around the blocking protrusion (313) and the proximal end (311) of the push rod (31), so the piston (32) is mounted on the proximal end (311) of the push rod (31) securely. With further reference to FIG. 8, the piston (32′) can also be integrally formed on the push rod (31′) to form an integral plunger.

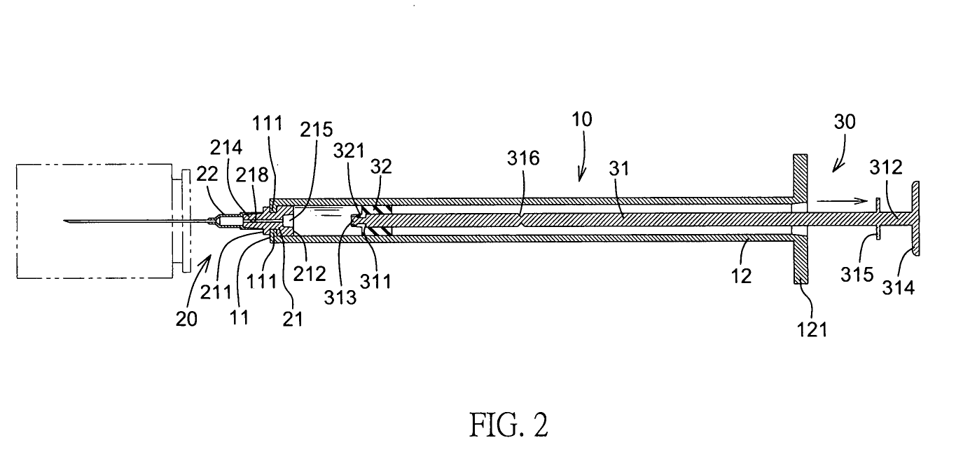

With reference to FIG. 2, when the safety syringe in accordance with the present invention is used to draw drug, the needle (22) is inserted into a bottle and the plunger (30) is pulled back to draw drug into the barrel (10) through the needle (22), the passage (218) and the recess (215) of the hub (21). Then the plunger (30) is pushed slightly to remove bubbles.

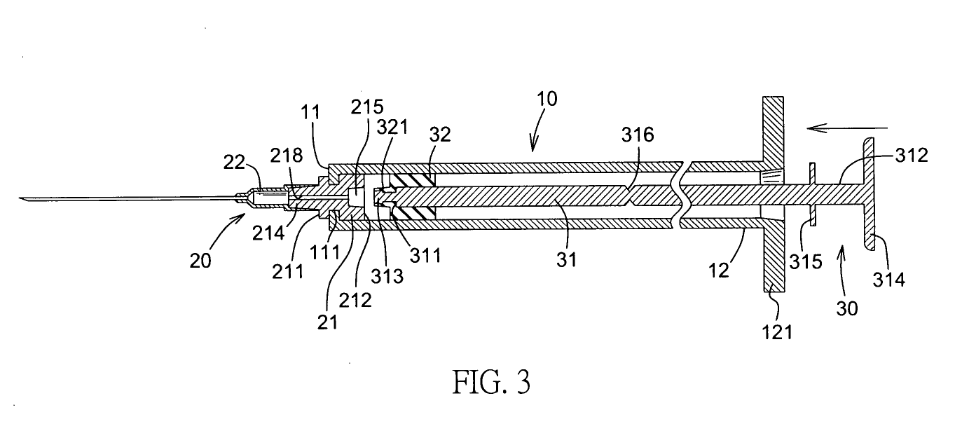

With reference to FIG. 3, when the safety syringe in accordance with the present invention is used to inject drug, the needle (22) is inserted into an injection site and the plunger (30) is pushed. Drug flows through the recess (215) and the passage (218) of the hub (21), the needle (22) and into the injection site.

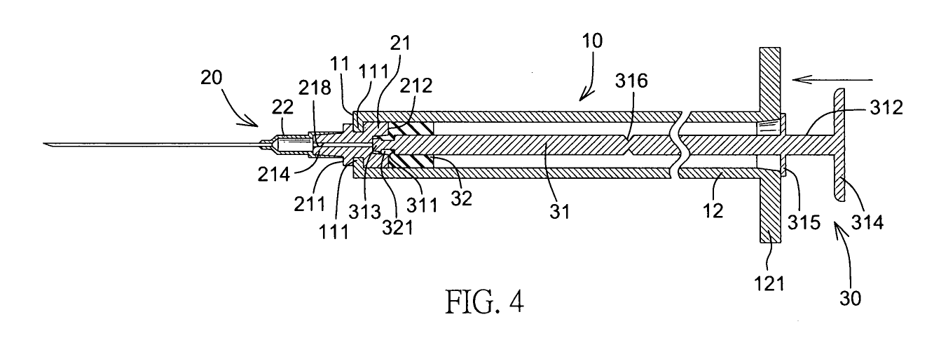

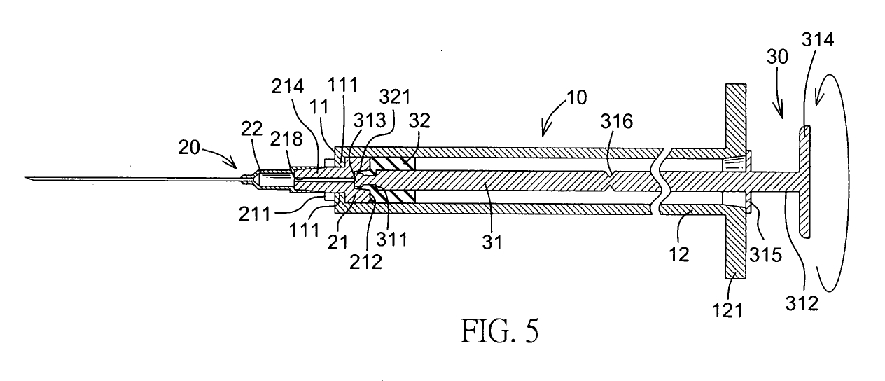

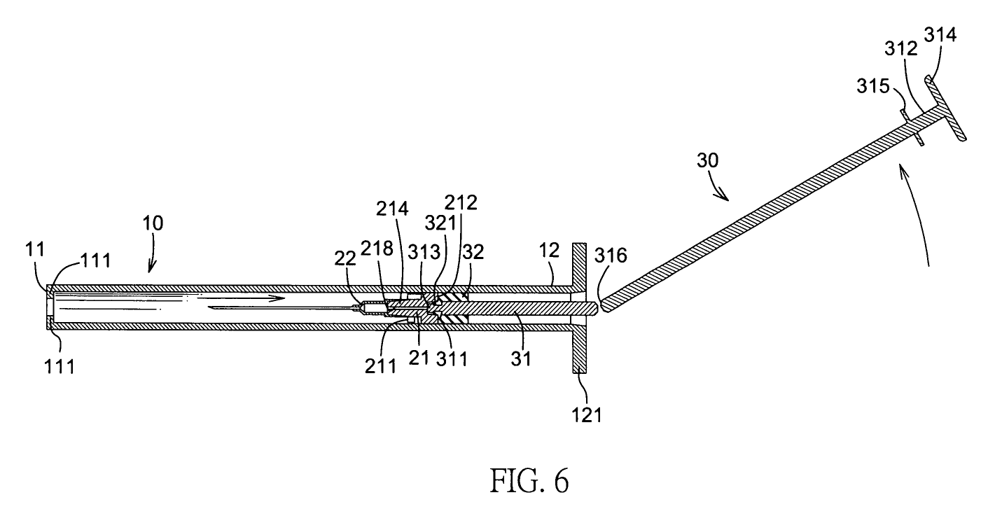

With reference to FIGS. 4, 5 and 6, after the safety syringe is used, the plunger (30) is pushed till the piston (32) abuts against the hub (21). At this time, the protrusion (321) of the piston (32) is inserted into the recess (215) of the hub (21). Then the plunger (30) is rotated and the hub (32) is loosened from the proximal open end (11) of the barrel (10) by the engagement between the protrusion (321) and the recess (215). After the hub (21) is loosened, the plunger (30) is pulled back to retract the hub (21) and the needle (22) into the barrel (10). Finally, the push rod (31) is broken at the notch (316) and the safety syringe will be kept from hurting any person. In addition, the safety syringe in accordance with the present invention does not have an inner thread and is easy to be manufactured.

Even though numerous characteristics and advantages of the present invention have been set forth in the foregoing description, together with details of the structure and function of the invention, the disclosure is illustrative only. Changes may be made in detail, especially in matters of shape, size and arrangement of parts within the principles of the invention to the full extent indicated by the broad general meaning of the terms in which the appended claims are expressed.

What is claimed is:

1. A safety syringe comprising:
a barrel having
a proximal open end;
a distal open end;
an inner wall;
an inner diameter;
at least one protrusion being formed on the inner wall near the proximal open end of the barrel; and
a flange being formed on the distal open end of the barrel;
a needle assembly being mounted on the proximal open end of the barrel and comprising
a hub having;
a proximal end;
a distal end;
an outer surface;
at least one L-shaped groove being defined in the outer surface of the hub and respectively corresponding to and engaging with the at least one protrusion of the barrel;
a connecting stub being formed on the proximal end and protruded out of the proximal open end of the barrel;
a recess being defined in the distal end of the hub; and
a passage being defined longitudinally through the hub; and
a needle being attached on the connecting stub of the hub and communicated with the passage; and
a plunger being mounted slidably in the barrel and comprising
a push rod having
a proximal end;
a distal end; and
a piston being mounted on the proximal end of the push rod and having
a diameter being slightly larger than the inner diameter of the barrel;
an outer surface;
an end facing the hub;
a protrusion being formed on the end facing the hub and corresponding to the recess of the hub.

2. The safety syringe as claimed in claim 1, wherein the recess of the hub is conical.

3. The safety syringe as claimed in claim 2, wherein
the push rod further comprises a blocking protrusion formed on the proximal end of the push rod and having an enlarged end.

4. The safety syringe as claimed in claim 3, wherein
the piston further comprises a through hole defined through the piston and corresponding to and engaging with the blocking protrusion and the proximal end of the push rod.

5. The safety syringe as claimed in claim 4, wherein
the barrel has two protrusions opposite to each other; and
the hub has two L-shaped grooves respectively corresponding to and engaging with the protrusions on the barrel.

6. The safety syringe as claimed in claim 5, wherein the hub further comprises an annular lip formed on the outer surface of the hub.

7. The safety syringe as claimed in claim 6, the piston further comprises an annular shoulder formed on the outer surface of the piston.

8. The safety syringe as claimed in claim 7, wherein the push rod further comprises a stop and a finger tab separately formed on the distal end of the push rod.

9. The safety syringe as claimed in claim 8, wherein the push rod further comprises a notch defined in the push rod.

10. The safety syringe as claimed in claim 1, wherein
the barrel comprises two protrusions opposite to each other; and
the hub has two L-shaped grooves respectively corresponding to and engaging with the protrusions on the barrel.

11. The safety syringe as claimed in claim 1, wherein the hub further comprises an annular lip formed on the outer surface of the hub.

12. The safety syringe as claimed in claim 1, wherein the piston further comprises an annular shoulder formed on the outer surface of the piston.

13. The safety syringe as claimed in claim 1, wherein the push rod further comprises a stop and a finger tab separately formed on the distal end of the push rod.

14. The safety syringe as claimed in claim 1, wherein the push rod further comprises a notch defined in the push rod.

15. The safety syringe as claimed in claim 1, wherein the needle is integrally formed on the hub.

16. The safety syringe as claimed in claim 1, wherein the piston is integrally formed on the proximal end of the push rod.
